(19) United States
(12) Patent Application Publication
(10) Pub. No.: US 2006/0293683 A1
(43) Pub. Date: Dec. 28, 2006

(54) Device for lengthening bones or bone parts

(76) Inventor: Roman Stauch, Assamstadt (DE)

Correspondence Address: BACHMAN & LAPOINTE, P.C., 900 CHAPEL STREET, SUITE 1201, NEW HAVEN, CT, 06510, US

(21) Appl. No.: 10/551,862

(22) PCT Filed: Feb. 4, 2004
(86) PCT No.: PCT/EP04/01005

§ 371 Date: Sep. 30, 2005

(30) Foreign Application Priority Data
Apr. 16, 2003 (DE) 103 17 776.0

(51) Int. Cl. A61B 17/58

(52) U.S. Cl. 606/90

(57) ABSTRACT

A device which lengthens bones or bone parts, especially for segmental transportation, comprising at least two elements which can be displaced in relation to each other. At least one locking element can be axially displaced in or along a guiding element.

### BACKGROUND OF THE INVENTION

The invention relates to a device for lengthening bones or bone parts for transport of segments, with at least two elements which can be moved relative to one another, at least one locking element being able to move axially in or along a guide element, and the at least one locking element being driven axially movably in or along the guide element by means of at least one drive unit.

Such devices are known, for example, in the form of medullary nails made up of two elements, especially bushings, which are movable relative to one another and which can be moved away from one another, for example by means of electric or hydraulic drives, in order to lengthen a bone or to transport a segment.

A disadvantage of the conventional devices is that they provide an extremely small stroke for the segment transport and have an undesirably large installation length. They are also difficult to clean and to operate.

DE 197 08 279 A1 discloses a distraction system for long bones for segment transport, the inside of the medullary nail having a bushing for receiving a securing element for fixing a bone segment, into which bushing a drivable threaded spindle engages. In this way, the length of the segment transport is restricted with a limited cross-sectional size.

DE 195 27 822 C1 describes an intracorporeal medullary nail which is composed of two parts which are movable relative to one another.

A similar device for lengthening bones and tissues is described in U.S. Pat. No. 5,356,411 in which two parts of a distraction device are moved away from one another by means of a spindle.

U.S. Pat. No. 5,720,746 describes a mechanical device for displacing two elements relative to one another, a second element being able to be moved out from the first element.

EP 1 033 112 A2 discloses a medullary nail for bone distraction, in which a coil is integrated in a proximal end region, which coil allows the contactless feed of an electrically driven motor.

A purely mechanically driven distraction device is set forth in U.S. Pat. No. 5,704,938, in which one part of a distraction device can be moved out relative to the other part via a spindle.

It is an object of the present invention to make available a device for lengthening bones or bone parts which eliminates said disadvantages and with which a very considerable stroke is possible together with a limited installation size.

Such a device should also be easy to clean and to disinfect, and in particular also easy to disassemble. Moreover, this device should be able to be operated and controlled with precision.

### SUMMARY OF THE INVENTION

The object is achieved by the fact that the drive unit is formed by a motor element, if appropriate with downstream gear and control unit, and by a spindle element adjoining the motor element or the gear, the at least one lock sitting directly on the spindle element, and the spindle element being designed as a threaded rod which extends through the at least one lock and engages with it.

In the present invention, it has proven particularly advantageous to insert at least one locking element in an axially movable manner into a guide element or to guide it along a guide element. The embodiment is preferred in which the at least one locking element is inserted into a guide slot of a guide element and can be moved axially to and fro along this guide slot by means of a spindle element, threaded rod or the like. The spindle element is driven by means of a drive unit or a motor element, if appropriate with upstream gear, and moves the lock in an exact and precise axial movement to and fro in the guide slot. The lock, which if appropriate engages over the outside of the guide element, is used to receive a bone segment which has been separated from a bone or bone part. The bone segment which has been separated from the bone or bone part, and which is fixed on the locking element, is distracted from it, or moved to the opposite bone or bone part, preferably very slowly, for example at 0.5 mm to 1.5 mm a day, preferably at 1 mm a day, so that in this way it is possible to ensure transport of the segment and a restoration and growth of the bone through osteosynthesis.

It should also fall within the context of the present invention for the lock to engage, for example, on corresponding grooves or the like and be guided in these, and to be moved axially via a spindle element, a threaded rod, a cable or the like along the guide element for transport of the bone segment.

In a further illustrative embodiment of the present invention, it is also possible for two locking elements to sit on the spindle element and be guided along the guide element, preferably in the guide slot, so that, for example, at both ends of both opposite bone parts, a separated bone segment is secured on the locking element and, by suitable actuation of the drive unit or of the spindle element, the bone segments and locks are moved slowly toward one another in order to produce a complete bone.

### BRIEF DESCRIPTION OF THE DRAWINGS

Further advantages, features and details of the invention will become evident from the following description of preferred illustrative embodiments and from the drawing, in which:

FIG. 1a is a diagrammatic side view of a drive unit for a device for lengthening bones, for insertion into a guide element;

FIG. 1b is a diagrammatic plan view of the guide element;

FIG. 2 is a diagrammatic plan view of the device for lengthening bones, composed of guide element and inserted drive unit;

FIG. 3 is a diagrammatic side view of the device according to FIG. 2;

FIG. 4 is a diagrammatic plan view of the device according to FIG. 2 in one possible position of use;

FIG. 5 is a diagrammatic plan view of a further illustrative embodiment of the device according to FIG. 2;

FIG. 6 is a diagrammatic plan view of the device according to FIG. 5 in one possible position of use.

### DETAILED DESCRIPTION

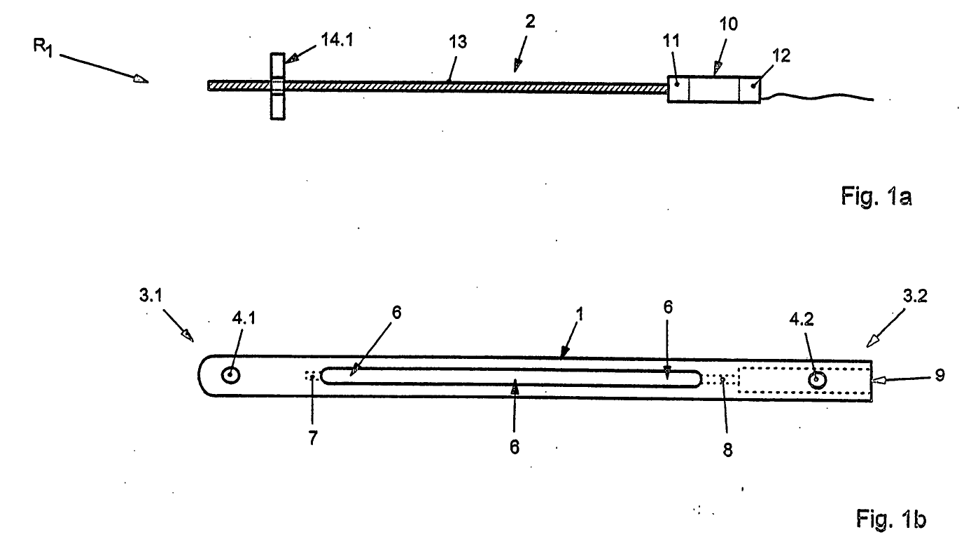

According to FIGS. 1a and 1b, a device R₁ according to the invention for lengthening bones or bone parts, in particular for transport of segments, comprises a guide element 1 and a drive unit 2. The guide element 1 can, for example, be designed as a medullary nail and is provided at each end area 3.1, 3.2 with through-openings 4.1, 4.2, respectively, which serve for fixing, in particular locking, the guide element 1 in or to a bone 5 or bone part. Securing elements (not shown in more detail here) fix the guide element 1 releasably in the bone 5.

Moreover, a preferably continuous and elongate guide slot 6 is provided in the axial direction in the guide element and has, at one end, a bearing recess 7, if appropriate designed also as a through-bore. At the other end, the guide slot 6 is adjoined by a passage 8 and, preferably following the latter in the end area 3.2, a receiving opening 9.

The drive unit 2 is preferably made up of a motor element 10 and, if appropriate, upstream gear 11 and downstream control unit 12.

The motor element 10 and/or gear 11 are adjoined by a drivable spindle element 13 on which at least one locking element 14.1 sits. The spindle element 13 can be designed, for example, as a threaded rod or the like and extends through the locking element 14.1 or engages with an internal thread of the locking element 14.1.

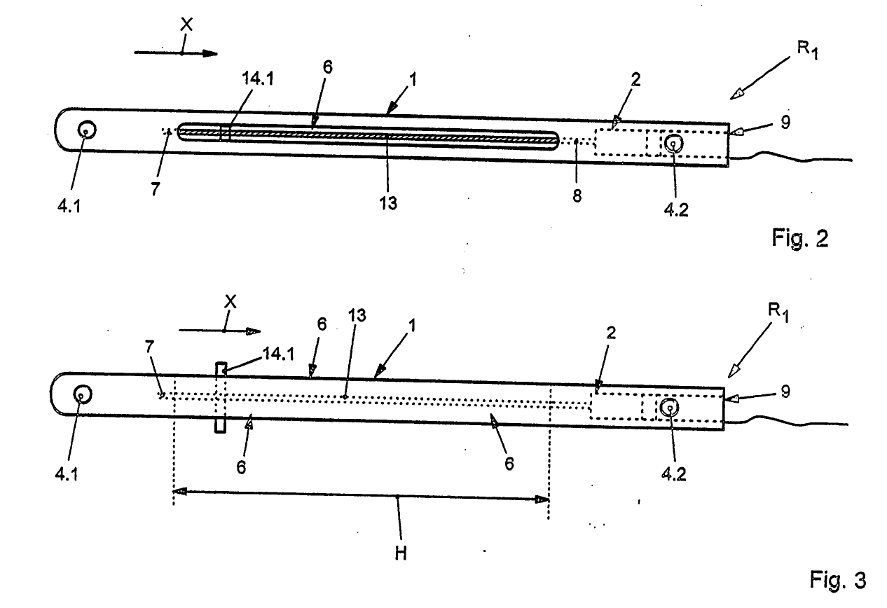

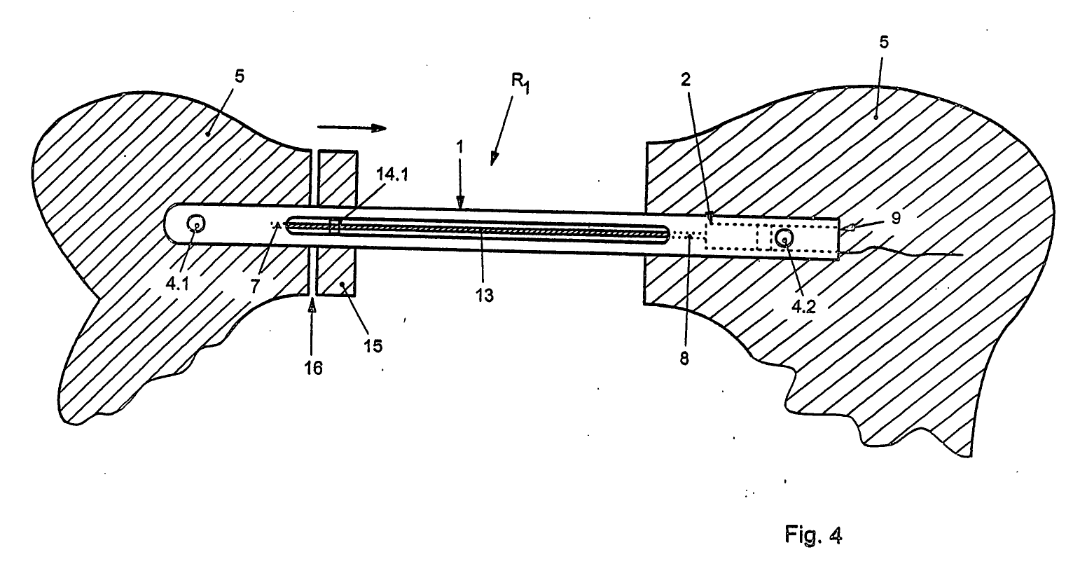

The plan view according to FIG. 2 shows the device R₁ with the drive unit 2 inserted into the guide element 1. The drive unit 2 can be inserted without locking element 14.1 through the receiving opening 9 of the guide element 1 and into the latter, the locking element 14.1 being pushed in through the guide slot 6 and being taken up by the suitably advanced spindle element 13, the end area of the spindle element 13 then coming to bear in the bearing recess 7.

The drive unit 2 can be fixed releasably in this position, in particular the motor element 10 in the receiving opening 9 of the guide element 1, and the drive unit 2 can be powered or controlled via connecting cables (not shown here), inductive adapters or the like. In this position of use, the spindle element 13 can be driven in rotation by suitable actuation of the motor element 10 or gear 11, so that, in this way, the locking element 14.1 can be driven or moved in or counter to an indicated direction X, the locking element 14.1 being guided through the guide slot 6 and executing only an axial movement in the indicated direction X.

As is shown in particular in the diagrammatic side view, the locking element 14.1 can be moved axially to and fro along the guide slot 6 depending on the driving direction of the spindle element 13.

The length of the guide slot 6 determines a stroke H by which the locking element 14.1 can be moved and driven.

The mode of operation of the present invention is as follows:

As is shown in FIG. 4, the device R₁ according to the invention is fitted, for example, into two end bones 5 or bone parts 5 which are intended to fuse back together by means of transport of a bone segment 15. A bone segment 15 is separated from one bone 5 in the area of a separating site 16 shown here. The bone segment 15 is then connected to the lock 14.1 or engages in the latter or is fixed thereon by means of securing elements. It is also conceivable that the locking element 14.1 for transport of the bone segment 15 is arranged in the area of the separating site 16 and engages behind the bone segment 15. The spindle element 13 is then activated, for example at selected time intervals, by means of the drive unit 2 via the motor element 10 and slowly moves the locking element 14.1 and thus the bone segment 15 to the opposite bone 5, for example at 0.5 mm to 1.5 mm a day, preferably at 1 mm a day.

In this way, the bone 5 can be lengthened again or can be fully restored by segment transport.

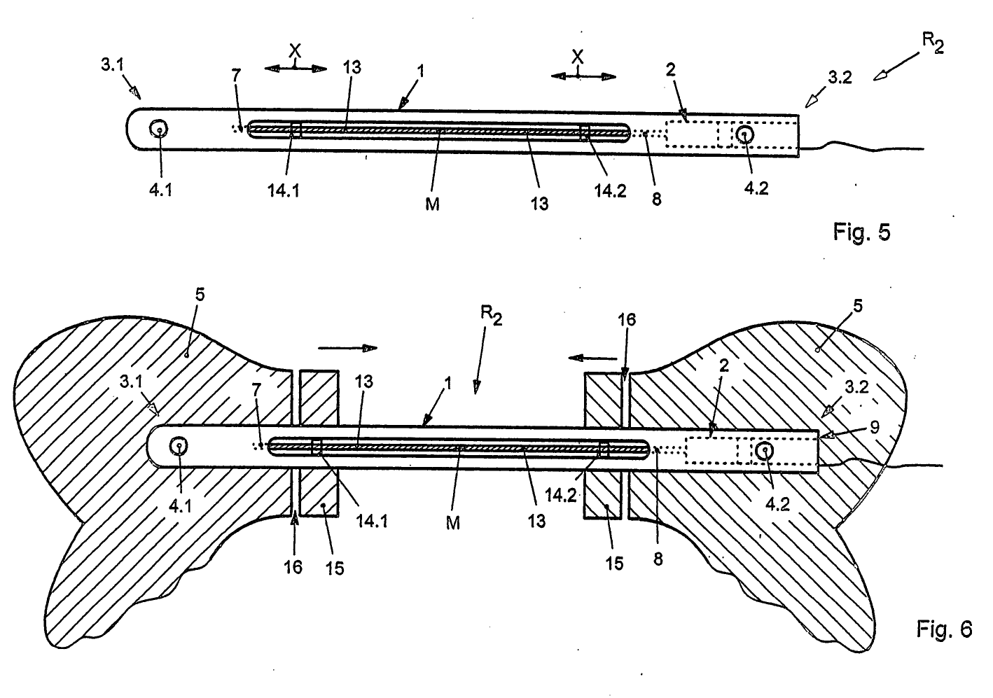

In a further illustrative embodiment of the present invention according to FIG. 5, a device R₂ is used which corresponds substantially to the aforementioned type. The difference here is that the locking element 14.1 sits on the spindle element 13 near the end area 3.1 and a further locking element 14.2 sits on it near the end area 3.2.

The thread turns of the spindle element are preferably designed in one half as right-hand threads, for example, and in the other half as left-hand threads, for example, so that the locking elements 14.1, 14.2, which are preferably at an identical distance from a middle M, can be moved uniformly toward one another or away from one another by the driving movement of the spindle element 13.

In this way, for example, segments 15 from each bone 5 or bone part can be moved toward one another, so that, for example, with an advance movement of 1 mm a day, a segment transport of two bone parts can be considerably accelerated. This is also intended to lie within the scope of the present invention.

1-14. (canceled)

15. A device for lengthening bones (5) or bone parts, comprises at least two elements which can be moved relative to one another, and including at least one locking element (14.1, 14.2) axially movable in or along a guide element (1).

16. The device as claimed in claim 15, further including means for moving the at least one locking element (14.1, 14.2) in or along the guide element (1).

17. The device as claimed in claim 16, wherein said means comprises at least one drive unit (2).

18. The device as claimed in claim 16, wherein the guide element (1) has an elongate, continuous guide slot (6).

19. The device as claimed in claim 16, wherein the guide element (1) comprises, at each end, a radial through-openings (4.1, 4.2) for the passage and engagement of securing elements for fixing the guide element (1) in the bone (5) or bone parts.

20. The device as claimed in claim 16, wherein the guide element (1) comprises, at one end, a receiving opening (9) for the reception and engagement of a drive unit (2).

21. The device as claimed in claim 17, wherein the drive unit (2) is formed by a motor element (10), with downstream gear (11) and control unit (12), and by a spindle element (13) adjoining the motor element (10) or adjoining the gear (11), wherein the at least one lock (14.1, 14.2) sits on the spindle element (13).

22. The device as claimed in claim 21, wherein the spindle element (13) comprises a threaded rod which passes through the at least one lock and engages with the at least one lock.

23. The device as claimed in claim 22, wherein the drive unit radial turns the spindle element (13) or threaded rod, wherein the locking element (14.1, 14.2) inserted into the guide slot (6) is moved axially to and fro along the guide element (1).

24. The device as claimed in claim 18, wherein the locking element (14.1, 14.2) comprises a rectangular or round cross section and engages at least partially over an outside of the guide slot (6) of the guide element (1).

25. The device as claimed in claim 21, wherein the bone segment (15), can be moved via the locking element (14) by means of the spindle element (13) being driven by the drive unit, wherein a separating site (16) is formed between a bone part and the bone segment (15).

26. The device as claimed in claim 25, wherein the locking element (14.1, 14.2) engages in the bone segment (15).

27. The device as claimed in claim 20, wherein the drive unit (2) is pushed axially into the receiving opening (9), and a motor element (10) is fitted against rotation in the guide element (1) in the area of the receiving opening (9).

28. The device as claimed in claim 27, wherein at one end of the guide slot (6), there is a recess (7) for bearing a spindle element (13).

29. The device as claimed in claim 28, wherein two locking elements (14.1, 14.2) sit on the spindle element (13) and, upon actuation of the motor element (10), are driven toward or away from one another in the guide slot (6) of the guide element (1).
